(19) United States
(12) Patent Application Publication
(10) Pub. No.: US 2025/0171607 A1
(43) Pub. Date: May 29, 2025

(54) DEPOLYMERIZATION OF POLYURETHANES WITH ORGANIC AMINE BASES

(71) Applicant: Evonik Operations GmbH, Essen (DE)

(72) Inventors: Jens Hildebrand, Johannesberg (DE); Annegret Terheiden, Alpen (DE); Natalia Hinrichs-Tontrup, Alzenau (DE); Juan Jesus Burdeniuc, Colmar, PA (US); Emily Clare Schweissinger, Dortmund (DE)

(73) Assignee: Evonik Operations GmbH, Essen (DE)

(21) Appl. No.: 18/839,819

(22) PCT Filed: Feb. 22, 2023
(86) PCT No.: PCT/EP2023/054375

§ 371 Date: Aug. 20, 2024

(30) Foreign Application Priority Data
Feb. 22, 2022 (EP) 22157887.5

(51) Int. Cl. C08J 11/14; C08G 18/24; C08G 18/42; C08G 65/48; C08J 11/28

(52) U.S. Cl. CPC C08J 11/14; C08G 18/244; C08G 18/4244; C08G 65/48; C08J 11/28; C08G 2110/0083; C08J 2375/14

(57) ABSTRACT

A hydrolyzing process improves the depolymerization of polyurethanes by contacting the polyurethane with water in the presence of an organic amine base and a phase transfer catalyst. The mild reaction conditions and low salt concentration in the reaction mixture allow to increase the yields of polyether polyol and organic polyamines, respectively.

The present invention relates to an improved process for depolymerization of polyurethanes under mild conditions and low salt concentration in the reaction mixture, wherein polyether polyols and polyamines can be recovered in high yields.

Polyurethanes are materials of considerable utility in the production of rigid and flexible foams, solid and microcellular elastomers, sealants, coatings and adhesives. The versatility, relatively low cost, and superior properties of polyurethanes have resulted in the rapid growth of the polyurethane industry over the past 50 years. Currently, many thousand tons of polyurethanes are produced each year throughout the world. Unfortunately, most polyurethanes are thermoset materials which are cross-linked to one degree or another. Unlike thermoplastics such as polyethylene, polypropylene, and polystyrene, scrap or waste polyurethanes thus cannot be readily remelted or reprocessed into useful articles. Since it would be highly desirable for economic and environmental reasons to reuse or recover the large volume of scrap or waste polyurethane generated each year rather than burning it or disposing of it in landfills, considerable inventive effort has been devoted to devising processes for recovering useful chemical components from scrap polyurethane materials.

Glycolysis, as for example disclosed in EP 0 835 901 A2, is used for recycling, i.e. depolymerization, of PUs (polyurethanes) waste including both rigid and flexible type products. These methods require various steps like (1) grinding, (2) step-wise addition of waste to diethylene glycol in the presence of catalyst, (3) alkoxylation, and (4) degassing and filtration for recovering polyols.

Ammonolysis and aminolysis are alternative methods to recycle scraps of polyurethane. Aminolysis results in the formation of ureas rather than of amines. To obtain amines, hydrolysis of the ureas has to be carried out in a second step. Said process is complex, requires high temperatures and has insufficient yields. U.S. Pat. No. 6,515,036, for example, discloses a two-step process, wherein aminolysis of polyurethane with aromatic amines, without addition of water, is carried out as step 1 to form a urea. Thereafter, in step 2 hydrolysis of the urea with water at temperatures above 200° C., without amine being present during hydrolysis, is carried out. U.S. Pat. No. 3,117,940 discloses aminolysis of polyurethane with amines having primary amino groups to obtain ureas. No hydrolysis is described. DE 102006036007 A1 also describes a process wherein polyurethanes are recycled via aminolysis. Further examples of aminolysis processes are disclosed in EP 0 990 674 A2 and DE 42 17 524 A1. Z. Dai, “Effect of diaminotoluene on the decomposition of polyurethane foam waste in superheated water”, Polymer Degradation and Stability (2002), 76 (2), 179-184, discloses a process for hydrolysis of polyurethanes in supercritical water at reaction temperatures above 250° C. with and without aromatic amine as base catalyst. G. Campbell, “Polyurethane waste disposal process development: amine recovery”, Journal of Applied Polymer Science (1977), 21 (2), 581-4, discloses a hydrolysis process of polyurethane with gaseous azeotrope water/aniline at temperatures above 200° C. The processes of Dai and Campbell are without commercial relevance because of the reaction conditions and high equipment costs.

Prior art processes disclosed in EP 0 990 674 A2 and DE 42 17 524 A1 are two step processes. In EP 0 990 674 A2 polyurethane is reacted in a first step with a solubilizing agent containing a polyamine compound, a low molecular glycol or an amino alcohol, i.e. aminolysis or alcoholysis is conducted as first step. During this first step polyurethane is dissolved and urethane as well as urea linkages are cleaved in the polyurethane. The resulting solution is thereafter subjected to hydrolysis in a second step, i.e. water is added after all polyurethane has been dissolved and no polyurethane is present anymore. In DE 42 17 524 A1 polyurethane is converted via aminolysis, by reacting with an amine and a glycol, into polyurethane urea in a first step. Water is added in a second step after all polyurethane has been converted to polyurethane urea. Two step processes need to be carried out batch-wise. Step one needs to be finished before the second step can be carried out. They are inefficient in view of room time-yield.

US20100093880 discloses a process to convert polyurethane to polyol and isocyanate rather than polyol and amine. In a first step aminolysis in an inert solvent, i.e. a solvent being free of water, is carried out to form secondary bisureas. Said secondary bisureas are cleaved in a subsequent step to form isocyanates.

Acidolysis has also been suggested to recycle polyols. None of these processes has been used in large industrial scale, yet. They are too complex, expensive, i.e. require high temperatures and pressures, and the quality of the recycling polyols low, so that only small amounts can be used together with large amounts of virgin raw materials to produce new polyurethane foams.

Hydrolysis methods were also tested for depolymerization of polyurethanes in the prior art, for example in U.S. Pat. No. 4,196,148 A and CN 110105621 B. Hydrolysis of a polyurethane using base catalysis to recover polyether polyols and polyamines as known in the art, however, suffers from several disadvantages. At relatively low temperatures, the hydrolysis rate is slow, respectively the hydrolysis is incomplete. At higher temperatures, the rate is faster but certain undesired side reactions may occur. U.S. Pat. No. 5,208,379 for example suggests a method to hydrolyze a polyurethane produced by reacting an active hydrogen-containing polyether and an organic polyisocyanate, which comprises contacting said polyurethane with water in the presence of an effective amount of a strong base selected from the group consisting of alkali metal oxides, alkali metal hydroxides, alkaline earth metal oxides, and alkaline earth metal hydroxides and an effective amount of an activating agent selected from the group consisting of quaternary ammonium salts containing at least 15 carbon atoms and organic sulfonates containing at least 7 carbon atoms for a time and at a temperature effective to yield the active hydrogen-containing polyether and an organic polyamine. The process of U.S. Pat. No. 5,208,379 is disadvantageous because of the very corrosive conditions applied. Special, very expensive equipment would be needed for implementation in a commercial plant. Even though U.S. Pat. No. 5,208,379 discloses in the general description that the reaction temperature may be chosen in a range of from 80 to 225° C., example 19 shows that at 120° C. only partial hydrolysis took place and example 18 shows that at 140° C. yields were only 70%. Thus, the process of U.S. Pat. No. 5,208,379 cannot economically be used at lower temperatures. In addition, the process of U.S. Pat. No. 5,208,379 requires use of large amounts of inorganic bases. Such process is problematic for sustainability and may cause problems in downstream processing if water is removed from the reaction solution. Said water removal increases the base concentration and the alkalinity of the reaction solution. Due to higher alkalinity of the reaction solution the risk of side reactions increases. In the process of CN 110105621 B, the recycled amine, i.e. the aromatic polyamine, is separated from the recycled polyol via precipitation of the recycled amine. This process is inefficient because it requires large amounts of water to precipitate the recycled amine. As will be shown in the comparison examples below, the process of CN 110105621 B produces unwanted by-products and can be operated batch-wise only.

Therefore, a strong need exists to provide more efficient and sustainable processes for polyurethane recycling to recover polyether polyols and/or polyamines in good quality, good yield.

Therefore, subject of the present invention was to provide a new process for depolymerization of polyurethanes that overcomes the aforementioned deficiencies of prior art methods.

A particular subject of the invention was to provide a process that can be carried out in standard equipment, i.e. steel reactors.

Another specific problem of the invention was to provide a process that can be operated at lower temperatures compared to the prior art methods, with good yields.

Another specific problem of the invention was to provide a hydrolysis process for polyurethane, which allows separation of the base used in the process from polyols and polyamines under mild conditions.

In another special problem of the invention was to provide a process with less side reactions compared to polyurethane hydrolysis processes using strong inorganic bases.

A further specific problem to be solved by the invention was to provide a process that allows to obtain polyether polyols and/or polyamines in a quality very close to that of the raw materials used to produce the polyurethane that shall be recycled. It should be possible to use recovered polyether polyols and/or polyamines in high proportions for production of new polyurethanes.

Another special problem of the invention was to provide a process being beneficial compared to the prior art in view sustainability, in particular in view of a reduction or avoidance of inorganic salt waste.

Further problems solved by the present invention but not described before, result from the subsequent description, examples and claims.

The inventors surprisingly found out that a method of hydrolyzing a polyurethane, preferably polyurethane produced by reacting an active hydrogen containing polyether and an organic polyisocyanate, which comprises contacting said polyurethane with water in the presence of an organic amine base or preferably with water in the presence of an organic amine base and a phase transfer catalyst, wherein
the organic amine base is an aliphatic amine, preferably having a boiling point below that of at least one, more preferred more than one, even more preferred all organic polyamines produced as product of the polyurethane hydrolysis, and
the reaction mixture comprising the polyurethane, water and the organic amine base is a stirred homogeneous or heterogeneous mixture, preferably a solution or an emulsion or a dispersion or combinations thereof, during hydrolysis, and
wherein the organic amine base is separated from the active hydrogen containing polyether, preferably polyether polyol, and the organic polyamines formed during polyurethane hydrolysis via distillation or extraction or membrane filtration, allows to obtain an active hydrogen containing polyether, preferably polyether polyol, and an organic polyamine in high yields.

In a preferred embodiment of the invention a phase transfer catalyst, even more preferably a phase transfer catalyst selected from the group consisting of quaternary ammonium salts containing 6 to 30 carbon atoms and organic sulfonates containing at least 7 carbon atoms is added to the reaction mixture during hydrolysis. This allows to increase the yields of polyether, preferably polyether polyol, and organic polyamine.

A wide variety of organic amine bases, that can easily be separated from the polyol and polyamine components of the hydrolyzed polyurethane, preferably by distillation or extraction, can be used in the process of the invention to effectively depolymerize polyurethanes.

In contrast to the inorganic bases used in for example U.S. Pat. No. 5,208,379, the preferred organic amine bases used in the present invention are non-ionic organic bases, i.e. are not in a salt form. Thus, the amounts of salts in the reaction solution, that needs to be separated and disposed was significantly reduced and sustainability of the process increased. The inventors surprisingly found out, that the organic amine bases used in the present invention can be used without addition of a phase transfer catalyst, i.e. without addition a quaternary ammonium salt used as phase transfer catalyst in U.S. Pat. No. 5,208,379. This allows a further reduction of the salt load of the reaction mixture and provides additional ecological and economic benefits.

In contrast to strong inorganic bases, the organic amine bases used in the present invention are non-corrosive, thus the process of the invention can be carried out in standard equipment under low or non-corrosive conditions.

The process of the invention is very flexible regarding to the use of phase transfer catalysts. Inventors found out, that if a phase transfer catalyst is used, it is preferred to use ammonium cations. Ammonium cations with a low number of carbon atoms, i.e. below 15, can be effectively used as well as such with a higher number of carbon atoms, i.e. 15 to 30. Use of such phase transfer catalysts allows to increase the polyol yield and increases flexibility with regard to the reaction temperature.

Recovered active hydrogen containing polyether and/or organic polyamines of the present invention are of excellent quality and can be used in high proportions to produce new polyurethane foams. Even if 100% active hydrogen containing polyether of the present invention are used to produce new polyurethane foams high quality polyurethane foams could be obtained. Without being bond to any theory inventors believe, that the specific mild reaction conditions and the use of organic amines as bases in the process of the invention avoids the formation of by-products which could cause problems during polyurethane production. An indicator for reduced amounts of side-products is the color index of the recovered polyether and/or polyamine.

In the process of the invention the organic amine base is separated from the active hydrogen containing polyether, preferably polyether polyol, and the organic polyamines formed during polyurethane hydrolysis via distillation or extraction or membrane filtration. Inventors found out that in particular distillation and extraction are most efficient to ensure a high recovery rate of the catalyst, which is preferably re-used in the process of the invention. By separation of the catalyst from the reaction mixture obtained after hydrolysis, i.e. without prior separation of other components of the reaction mixture, the contact time of catalyst and the other components could be minimized, and side reaction could be avoided.

An embodiment of the present invention therefore is a process as defined in claim 1, the dependent claims and the description.

Before describing the invention in more details some important terms are defined as follows:

The verb “to comprise” as is used in the description, examples and the claims and its conjugation is used in its non-limiting sense to mean that items following the word are included, but items not specifically mentioned are not excluded. “Comprising” includes “consisting of” meaning that items following the word “comprising”, are included without any additional, not specifically mentioned items, as preferred embodiment.

Reference to an element be the indefinite article “a” or “an” does not exclude the possibility that more than one of the elements is present, unless the context clearly requires that there is one and only one of the elements. The indefinite article “a” or “an” thus usually means “one or more”.

The terms “catalyst” and “activating agent” are used synonymously in the present invention.

Polyurethane (PU) in the context of the present invention is especially understood to mean a product obtainable by reaction of polyisocyanates and polyols, or compounds having isocyanate-reactive groups. The polyurethanes which may be subjected to the process of the present invention are preferably those prepared from active hydrogen-containing polyethers and polyisocyanates. Polyurethanes of this type are well known and are described, for example, in Ulrich, “Urethane Polymers”, in Encyclopedia of Chemical Technology, Vol. 23, pp. 576-608 (1983) and Backus et al., “Polyurethanes”, in Encyclopedia of Polymer Science and Technology, Vol. 13, pp. 243-303 (1988). Any known polyurethane can be used in the process of the invention.

The active hydrogen-containing polyether preferably is a polyether polyol (i.e., a polyether having primary and/or secondary end groups, preferably hydroxyl groups) but may also be an amine-functionalized polyether (e.g., the “Jeffamine” polyoxypropylamines sold by Texaco Chemical Co.). Such materials are generally made by the catalytic ring-opening polymerization of one or more cyclic ethers such as epoxides, oxetanes, or oxolanes. Initiators having two or more active hydrogens such as polyhydric alcohols, amines, or acids may be employed to vary the functionality (number of active hydrogens) of the polyether. If more than one type of cyclic ether is used, they may be reacted either simultaneously (to yield a random-type copolymer) or sequentially (to yield a block-type copolymer). Illustrative cyclic ethers include propylene oxide, ethylene oxide, butylene oxide, tetrahydrofuran, and oxetane. Examples of suitable active hydrogen-containing polyethers include polypropylene glycol, polyethylene glycol, polytetramethylene glycol, polytrimethylene glycol, ethylene oxide-capped polypropylene glycol, random copolymers of ethylene oxide and propylene oxide.

The structure of the active hydrogen containing polyether, preferably polyether polyols, recovered in the process of the invention correlates to the structure of the active hydrogen containing polyether used to prepare the polyurethane to be treated in the process of the invention.

The structure of the polyamines recovered in the process of the invention correlates to the structure of the polyisocyanates used to prepare the polyurethane to be treated in the process of the invention. “Polyamines” as used in the present invention includes diamines and preferably includes amines having two or more primary amino groups in the molecule.

The polyurethane employed in the process of this invention may be derived from any polyisocyanate reactant (i.e., an organic compound containing two or more isocyanate groups). Suitable polyisocyanates include, but are not limited to, aliphatic diisocyanates, cycloaliphatic diisocyanates, aryl alkyl diisocyanates, aromatic diisocyanates (e.g., toluene diisocyanates and diisocyanatodiphenyl methanes), aromatic triisocyanates, as well as isocyanate mixtures such as the isocyanates commonly referred to as “PMDI”. Modified, masked, or blocked polyisocyanates may, of course, also be utilized.

The polyurethane used is the process of the present invention may also include any of the conventional additional reactants or additives known in the art such as for example chain extenders or curatives (relatively low molecular weight active hydrogen-containing compounds such as glycols and di- or polyamines), physical or chemical blowing agents, flame retardants, surfactants, fillers, stabilizers, anti-oxidants, colorants, polymers other than the polyurethane polymer (e.g., styrene-acrylonitrile copolymers such as are found in polymer polyols), catalysts, for example catalysts promoting the gelling reaction (isocyanate-polyol), the blowing reaction (isocyanate-water) and/or the dimerization or trimerization of the isocyanate. The polyurethane may be in solid, microcellular, or foam form and may range from a rubbery, elastomeric, flexible material to a hard, rigid substance.

To facilitate handling of the polyurethane, it is preferably desirable to chop, pulverize, grind, or otherwise comminute the polyurethane such that it is in the form of relatively small particles or granules. If the polyurethane is a foam, it may be partially or fully compressed prior to contacting with the water and the organic amine base. If the polyurethane is in solid form, an initial pulverization step is highly advantageous so as to maximize the surface area available for reaction (thereby reducing the reaction time required to achieve the desired level of hydrolysis).

The process of this invention will result in the effective hydrolytic cleavage of the urethane and urea bonds present in the polyurethane being treated so as to generate active hydrogen containing polyether, in particular polyether polyols, polyamines, and, if the polyurethane was prepared using chain extenders or curatives, low molecular weight glycols, diols, diamines.

The organic amine bases used in the present invention are aliphatic amines, preferably having a boiling point below that of at least one, preferably more than one, more preferred all organic polyamines produced as product of the polyurethane hydrolysis.

Use of aliphatic organic amine bases allow to reduce reaction time and temperature compared to use of aromatic amines without decrease of polyol yield.

Preferably, the organic amine bases are used as non-ionic organic bases in the process of the invention. “Non-ionic” means that the base is not in the form of a salt before being added to the reaction mixture, i.e. does not comprise an anion and a cation. “Organic amine bases” are compounds which, in addition to carbon and hydrogen contain nitrogen and react to salt-like compounds with acids. Preferably the “organic amine bases” comprise one or more CH bonds.

The organic amine base used in the present invention preferably comprises one or more than one nitrogen atom(s) and more preferred is different from 1,4-dimethylpiperazin. Said nitrogen atoms may be primary, i.e. NH₂R, secondary, i.e. NHR₂, and/or tertiary, i.e. NR₃, wherein R being an alkyl group. Preferably, the organic base comprises one or more than one tertiary nitrogen atom(s). Without being bound to any theory, applicants believe that tertiary nitrogen atoms do not form ureas when reacting with the polyurethane and thus, catalyze direct hydrolysis of the polyurethane compared to primary or secondary amino groups.

Preferred organic amine base(s) is/are selected from the group consisting of
A base according to Formula (1)

(((R³)₂N—R²)—(O—R¹)ₓ)_{y}—N(R⁴)_{z} (1)
wherein
the R¹ groups in the molecule may be identical or different, the R² groups in the molecule may be identical or different, the R³ groups in the molecule may be identical or different, and the R⁴ groups in the molecule may be identical or different and wherein
R¹ are identical or different and are independently from each other selected from the group consisting of linear or branched alkylene radicals with 1 to 10, preferably 2 to 6, more preferred 2 to 4 carbon atoms, most preferred ethylene, propylene or isopropylene
R² are identical or different and are independently from each other selected from the group consisting of linear or branched alkylene or hydroxy alkylene radicals with 1 to 20, preferably 1 to 18, more preferred 2 to 6, even more preferred 2 to 4 carbon atoms and if R² is hydroxy alkylene R² comprises 1 to 5, preferably 1, 2 or 3, more preferred 1 or 2 and most preferred 1 hydroxy group(s), most preferred ethylene, propylene, butylene, hexamethylene, 2-hydroxypropylene or isopropylene
R³ are identical or different and are independently from each other selected from the group consisting of hydrogen, linear or branched alkyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O)ᵤH, (CH₂CH₂CH₂O)ᵥH and (CH₂CH(CH₃)CH₂O)_{w}H, most preferred hydrogen, methyl, ethyl, propyl, isopropyl, hydroxymethyl, 2-hydroxyethyl, 3-hydroxypropyl, 2-hydroxypropyl, 2-hydroxyisopropyl,
R⁴ are identical or different and are independently from each other selected from the group consisting of hydrogen, linear or branched, cyclic or alicyclic alkyl groups with 1 to 20, preferably 1 to 18, more preferred 1 to 6, even more preferred 1 to 4, most preferred 1, 2 or 3 carbon atoms, linear or branched hydroxy alkyl groups with 1 to 6, preferably 1 to 4, more preferred 1, 2 or 3 carbon atoms and cycloalkyl residues having 6 to 18 carbon atoms, preferably 6 to 12, more preferred 6 to 10 and even more preferred 6 or 7 carbon atoms, (CH₂CH₂O)ᵤH, (CH₂CH₂CH₂O)ᵥH and (CH₂CH(CH₃)CH₂O)_{w}H, most preferred methyl, ethyl, propyl, butyl, pentyl, hexyl, heptyl, octyl, cyclohexyl, isopropyl, tertbutyl, cyclohexyl, methylcyclohexyl, 2-cyclohexyl-ethyl, hydroxymethyl, 2-hydroxyethyl, 3-hydroxypropyl, 2-hydroxypropyl, 6-hydroxyhexyl and 2-hydroxyisopropyl, and wherein
u=1 to 14, preferably 1 to 6
v=1 to 14, preferably 1 to 6
w=1 to 14, preferably 1 to 6
x=0 or 1
y=0 to 3
z=0 to 3, with the proviso that if z=3, one, preferably two, more preferred all three R⁴ are not hydrogen,
y+z=3
A base according to formula (2)

(((R⁶)₂N—R⁵)ₐ(H)_{b}N)_{d}—CZ—(N(R⁷)₂)_{c} (2)
wherein
the R⁵ groups in the molecule may be identical or different, the residues R⁶ groups in the molecule may be identical or different and the residues R⁷ groups in the molecule may be identical or different and wherein
R⁵ are identical or different and are independently from each other selected from the group consisting of linear or branched alkylene radicals with 1 to 10, preferably 2 to 6, more preferred 2 to 4 carbon atoms, wherein one or more CH₂ groups may be replaced by O to form ether bonds, preferably R⁵ is ethylene, propylene or isopropylene
R⁶ are identical or different and are independently from each other selected from the group consisting of hydrogen, linear or branched alkyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O)ᵤH, (CH₂CH₂CH₂O)ᵥH and (CH₂CH(CH₃)CH₂O)_{w}H, most preferred hydrogen, methyl, ethyl, propyl, isopropyl, hydroxymethyl, 2-hydroxyethyl, 3-hydroxypropyl, 2-hydroxypropyl, 2-hydroxyisopropyl,
R⁷ are identical or different and are independently from each other selected from the group consisting of hydrogen, linear or branched alkyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O) UH, (CH₂CH₂CH₂O) vH and (CH₂CH(CH₃)CH₂O) wH, most preferred hydrogen, methyl, ethyl, propyl, isopropyl, and wherein
Z═O or NH,
u=1 to 14, preferably 1 to 6
v=1 to 14, preferably 1 to 6
w=1 to 14, preferably 1 to 6
a=0, 1 or 2
b=0, 1 or 2
a+b=2
c=0, 1 or 2
d=0, 1 or 2
c+d=2
cyclic or bicyclic, non-aromatic, nitrogen comprising organic base comprising 4 to 20 carbon atoms, preferably 5 to 14, more preferred 5 to 12 and most preferred 6 to 10 carbon atoms and 1 to 4 nitrogen atoms, preferably 1 to 3, more preferred 1, 2 or 3 nitrogen atoms, optionally the cyclic or bicyclic, non-aromatic, nitrogen comprising organic base comprises one or more O atoms and/or carries one or more substituents, such as linear or branched alkyl or alkenyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O) uH, (CH₂CH₂CH₂O) vH and (CH₂CH(CH₃)CH₂O) wH, most preferred hydrogen, methyl, ethyl, propyl, isopropyl, hydroxymethyl, 2-hydroxyethyl, 3-hydroxypropyl, 2-hydroxypropyl, 2-hydroxyisopropyl or one or more N and or O comprising functional groups and/or two or more cyclic or bicyclic non-aromatic, nitrogen comprising organic rings are bond to each other via alkylene or ether alkylene linkages with 1 to 12, preferably 1 to 6 carbon atoms, preferably the cyclic organic amine base is different from 1,4-dimethylpiperazin,
and mixtures thereof.

Preferably the amines are selected from the lists defined before such that they comprise one or more tertiary nitrogen atom(s) and that they are having a boiling point below that of at least one, preferably more than one, more preferred all organic polyamines obtained as product of the polyurethane hydrolysis.

Most preferred the organic amine base(s) is/are selected from the group consisting of
A trialkylamine according to Formula (3), as preferred embodiment of Formula (1),

NR⁴R⁴′R⁴″ (3)
with R⁴, R⁴′, R⁴″ are identical or different and are independently selected from the group consisting of hydrogen, linear or branched, cyclic or alicyclic alkyl groups with 1 to 20, preferably 1 to 18, more preferred 1 to 6, even more preferred 1 to 4, most preferred 1, 2 or 3 carbon atoms, linear or branched hydroxy alkyl groups with 1 to 6, preferably 1 to 4, more preferred 1, 2 or 3 carbon atoms and cycloalkyl residues having 6 to 18 carbon atoms, preferably 6 to 12, more preferred 6 to 10 and even more preferred 6 or 7 carbon atoms, (CH₂CH₂O)ᵤH, (CH₂CH₂CH₂O)ᵥH and (CH₂CH(CH₃)CH₂O)_{w}H, most preferred methyl, ethyl, propyl, butyl, pentyl, hexyl, heptyl, octyl, cyclohexyl, isopropyl, tertbutyl, cyclohexyl, methylcyclohexyl, 2-cyclohexyl-ethyl, hydroxymethyl, 2-hydroxyethyl, 3-hydroxypropyl, 2-hydroxypropyl, 6-hydroxyhexyl and 2-hydroxyisopropyl,
u=1 to 14, preferably 1 to 6
v=1 to 14, preferably 1 to 6
w=1 to 14, preferably 1 to 6,
with the proviso that one, preferably two, more preferred all three of R⁴, R⁴′ and R⁴″ are not hydrogen.
A polyamine according to Formula (4), as another preferred embodiment of Formula (1),

((R³)₂N—R²)₃N (4),
wherein
R² are identical or different and are independently from each other selected from the group consisting of linear or branched alkylene or hydroxy alkylene radicals with 1 to 20, preferred 1 to 18, more preferred 2 to 6, even more preferred 2 to 4 carbon atoms and if R² is hydroxyalkyl R² comprises 1 to 5, preferably 1, 2 or 3, more preferred 1 or 2 and most preferred 1 hydroxy group(s), most preferred ethylene, propylene or isopropylene
R³ are identical or different and are independently from each other selected from the group consisting of hydrogen, linear or branched alkyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O)ᵤH, (CH₂CH₂CH₂O)ᵥH and (CH₂CH(CH₃)CH₂O)_{w}H, most preferred hydrogen, methyl, ethyl, propyl, isopropyl, hydroxymethyl, 2-hydroxyethyl, 3-hydroxypropyl, 2-hydroxypropyl, 2-hydroxyisopropyl,
u=1 to 14, preferably 1 to 6
V=1 to 14, preferably 1 to 6
w=1 to 14, preferably 1 to 6
A polyamine according to Formula (5), as further preferred embodiment of Formula (1),

(((R³)₂N—R²)—(O—R¹)ₓ)_{y}—N(R⁴)_{z} (5)
wherein x=0 or 1, y=1 or 2 and z=1 or 2 and y+z=3 and wherein
R¹ are identical or different and are independently from each other selected from the group consisting of linear or branched alkylene radicals with 1 to 10, preferably 2 to 6, more preferred 2 to 4 carbon atoms, most preferred ethylene, propylene or isopropylene,
R² are identical or different and are independently from each other selected from the group consisting of linear or branched alkylene or hydroxy alkylene radicals with 1 to 20, preferably 1 to 18, more preferred 2 to 6, even more preferred 2 to 4 carbon atoms and if R² is hydroxyalkyl R² comprises 1 to 5, preferably 1, 2 or 3, more preferred 1 or 2 and most preferred 1 hydroxy group(s), most preferred ethylene, propylene, butylene, hexamethylene, 2-hydroxypropylene or isopropylene,
R³ are identical or different and are independently from each other selected from the group consisting of hydrogen, linear or branched alkyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O) UH, (CH₂CH₂CH₂O) vH and (CH₂CH(CH₃)CH₂O) wH, most preferred hydrogen, methyl, ethyl, propyl, isopropyl,
R⁴ are identical or different and are independently from each other selected from the group consisting of hydrogen, linear or branched, cyclic or alicyclic alkyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O)ᵤH, (CH₂CH₂CH₂O)ᵥH and (CH₂CH(CH₃)CH₂O)_{w}, most preferred methyl, ethyl, propyl, butyl, pentyl, hexyl, heptyl, octyl, cyclohexyl, isopropyl, tertbutyl, cyclohexyl, methylcyclohexyl, 2-cyclohexyl-ethyl, hydroxymethyl, 2-hydroxyethyl, 3-hydroxypropyl, 2-hydroxypropyl, 6-hydroxyhexyl and 2-hydroxyisopropyl
u=1 to 14, preferably 1 to 6
v=1 to 14, preferably 1 to 6
w=1 to 14, preferably 1 to 6
A polyaminoalkylurea according to Formula (6), as preferred embodiment of Formula (2),

(((R⁶)₂N—R⁵)ₐ(H)_{b}N)_{d}—CO—(N(R⁷)₂)_{c} (6)
wherein
R⁵ are identical or different and are independently from each other selected from the group consisting of linear or branched alkylene radicals with 1 to 10, preferably 2 to 6, more preferred 2 to 4 carbon atoms, wherein one or more CH₂ groups may be replaced by O to form ether bonds, preferably R⁵ is ethylene, propylene or isopropylene
R⁶ are identical or different and are independently from each other selected from the group consisting of hydrogen, linear or branched alkyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O)ᵤH, (CH₂CH₂CH₂O)ᵥH and (CH₂CH(CH₃)CH₂O)_{w}H, most preferred methyl, ethyl, propyl, isopropyl, hydroxymethyl, 2-hydroxyethyl, 3-hydroxypropyl, 2-hydroxypropyl, 2-hydroxy isopropyl,
R⁷ are identical or different and are independently from each other selected from the group consisting of hydrogen, linear or branched alkyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O)ᵤH, (CH₂CH₂CH₂O)ᵥH and (CH₂CH(CH₃)CH₂O)_{w}H, most preferred hydrogen, methyl, ethyl, propyl, isopropyl, hydroxymethyl, 2-hydroxyethyl, 3-hydroxypropyl, 2-hydroxypropyl, 2-hydroxyisopropyl, and wherein
u=1 to 14, preferably 1 to 6
v=1 to 14, preferably 1 to 6
w=1 to 14, preferably 1 to 6
a=0, 1 or 2
b=0, 1 or 2
a+b=2
c=0, 1 or 2
d=0, 1 or 2
c+d=2,

An organic base comprising a guanidino group according to Formula (7), as further preferred embodiment of Formula (2),

(((R⁶)₂N—R₅)ₐ(H)_{b}N)ₐ—C(NH)—(N(R₇)₂)_{c} (7)
wherein
R⁵ are identical or different and are independently from each other selected from the group consisting of linear or branched alkylene radicals with 1 to 10, preferably 2 to 6, more preferred 2 to 4 carbon atoms, wherein one or more CH₂ groups may be replaced by O to form ether bonds, preferably R⁵ is ethylene, propylene or isopropylene
R⁶ are identical or different and are independently from each other selected from the group consisting of hydrogen, linear or branched alkyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O)ᵤH, (CH₂CH₂CH₂O)ᵥH and (CH₂CH(CH₃)CH₂O)_{w}H, most preferred methyl, ethyl, propyl, isopropyl, hydroxymethyl, 2-hydroxyethyl, 3-hydroxypropyl, 2-hydroxypropyl, 2-hydroxyisopropyl,
R⁷ are identical or different and are independently from each other selected from the group consisting of hydrogen, linear or branched alkyl groups with 1 to 6, preferably 1 to 4, more preferred 1, 2 or 3 carbon atoms, linear or branched hydroxy alkyl groups with 1 to 6, preferably 1 to 4, more preferred 1, 2 or 3 carbon atoms, (CH₂CH₂O)ᵤH, (CH₂CH₂CH₂O)ᵥH and (CH₂CH(CH₃)CH₂O)_{w}H, most preferred methyl, ethyl, propyl, isopropyl, and wherein
u=1 to 14, preferably 1 to 6
v=1 to 14, preferably 1 to 6
w=1 to 14, preferably 1 to 6
a=0, 1 or 2
b=0, 1 or 2
a+b=2
c=0, 1 or 2, preferably 0 or 1, more preferred 0
d=0, 1 or 2, preferably 1 or 2, more preferred 2
c+d=2,
and mixtures thereof

Preferably the amines are selected from the lists defined before such that they comprise one or more tertiary nitrogen atom(s) and that they are having a boiling point below that of at least one, preferably more than one, more preferred all organic polyamines obtained as product of the polyurethane hydrolysis.

Most preferred organic amine bases are selected from the group consisting of triethylamine, tripropylamine, N,N dimethyl-N-propylamine, N, N-dimethyl-N-butylamine, N, N-dimethyl-N-pentylamine, N,N-dimethyl-N-hexylamine, N,N-dimethyl-N-cyclohexylamine, N, N-dimethyl-N-heptylamine, N,N-dimethyl-N-octylamine, N,N-diethyl-N-propylamine, N,N-diethyl-N-butylamine, N, N-diethyl-N-pentylamine, N,N-diethyl-N-hexylamine, N, N-diethyl-N-cyclohexylamine, N, N-diethyl-N-heptylamine, N,N-diethyl-N-octylamine, tetramethylethylenediamine (TMEDA), tetramethyl-1,3-propylenediamine (TMPDA), tetramethyl-1,4-butylenediamine (TMBDA), tetramethyl-1,6-hexamethylenediamine (TMHMDA), pentamethyldiethylenetriamine (PMDETA), N,N,N′N′-tetramethyl-bis(aminoethyl) ether, 1,4-diazabicyclo (2,2,2) octane (TEDA), trimethyl-triaza-cyclononane (TACN); dimethylethanolamine, dimethylaminoethoxyethanol, N,N-dimethylaminoethyl-N′-methyl-ethanolamine, tetramethylguanidine, N,N-bis(3-dimethylamino-propyl)-N-(2-hydroxypropyl) amine, N,N-dimethyl-N′,N′-bis(2-hydroxypropyl)-1,3-propylenediamine, dimethylaminopropylamine (DMAPA); N-methyl-N-2-hydroxypropyl-piperazine, bis(dimethylaminopropyl) amine, dimethylaminopropyl urea, N,N′-bis(3-dimethylaminopropyl) urea, 1,3-bis(dimethylamino)-2-propanol, 6-dimethylamino-1-hexanol, N,N′-bis(2-hydroxypropyl) piperazine, N-(2-hydroxypropyl)-morpholine, N-methyl-pyrrolidine, N-ethyl-pyrrolidine, N-(2-hydroxyethyl)-pyrrolidine, N-(2-hydroxypropyl)-pyrrolidine, N-propyl-pyrrolidine, N-allyl-pyrrolidine, N-methyl-piperidine, N-ethyl-piperidine, N-(2-hydroxyethyl)-piperidine, N-(2-hydroxypropyl)-piperidine, N-propyl-piperidine, N-allyl-piperidine and their mixtures, are used in the present invention.

Use of the bases described before allows to run the process of the invention in standard equipment, preferably in steel reactors, without special corrosion protection and thus, significantly contributes to a reduction of the invest costs for the plants. It is also possible to use very cheap bases that contribute to reduced operating costs.

The amount of organic amine base in the reaction mixture must be sufficient to catalyze the desired hydrolysis of the polyurethane at a practicable rate. Preferably the amine is used in stoichiometric amounts compared to the polyurethane or an excess of amine is used. More preferred the weight ratio of the sum of the organic amine bases to polyurethane is in the range of from 1:100 to 50:1, preferably 1:50 to 25:1, more preferred 1:10 to 20:1, even more preferred 1:5 to 10:1, and most preferred 1:2 to 3:1. Preferably the base is used in form of a base solution comprising a base and water, even more preferred as a saturated base solution.

Quaternary ammonium salts, organic sulfonates, or some combination or mixture thereof are preferably used as phase transfer catalysts in the process of the present invention. This allows to further increase the yields of the hydrolysis reaction. Most preferred quaternary ammonium salts are used.

Although the addition of even trace amounts of these phase transfer catalysts will accelerate the hydrolysis rate and increase the yields, it is preferred that at least 0.5 weight percent phase transfer catalyst based on the weight of the polyurethane be used, more preferably 0.5 to 15 weight percent, even more preferred 1 to 10 weight percent, particular preferred more 1 to 8 weight percent, especially preferred 1 to 7 and most preferred 2 to 6 weight percent.

The quaternary ammonium salts useful in the invention include those organic nitrogen-containing compounds in which the molecular structure includes a central positively-charged nitrogen atom joined to four organic (i.e., hydrocarbyl) groups, i.e. the ammonium cation, and a negatively charged anion such as halide, preferably chloride, bromide, hydrogen sulfate, alkyl sulfate, preferably methylsulfate and ethylsulfate, carbonate, hydrogen carbonate, carboxylate, preferably acetate, or hydroxide.

Quaternary ammonium salts are well known and are described, for example, in Cahn et al., “Surfactants and Defensive Systems”, in Encyclopedia of Chemical Technology, Third Edition Vol. 22, pp. 383-385 (1983) and Catonic Surfactants, E. Jungermann, Ed., Marcel Dekker, New York (1970), pp. 1-173. Many such compounds are commercially available at relatively low cost.

Quaternary ammonium salts containing an ammonium cation containing a total of 6 to 30 carbon atoms have been found to be most effective in the process of the invention. In contrast to the teachings of U.S. Pat. No. 5,208,379, the inventors found out that yields significantly decrease if ammonium cation containing a total of more than 30 carbon atoms are used. The same is true if the number of carbon atoms is below 6.

Phase transfer catalyst that have proven to be highly efficient and thus are preferably used in the process of the present invention are quaternary ammonium salts having the general structure R₁ R₂ R₃ R₄ NX wherein R₁, R₂, R₃, and R₄ are the same or different and are hydrocarbyl groups selected from alkyl, aryl, and arylalkyl and X is selected from the group consisting of halide, preferably chloride and/or bromide, hydrogen sulfate, alkyl sulfate, preferably methylsulfate and ethylsulfate, carbonate, hydrogen carbonate, carboxylate, preferably acetate, or hydroxide.

Preferably
R¹ and R² are the same or different and are alkyl groups with 1 to 12, preferably 1 to 10, more preferred 1 to 7, even more preferred 1 to 6, especially preferred 1 to 5 and most preferred 1 to 4 carbon atoms, wherein the alkyl groups may be linear, branched, cyclic, saturated or unsaturated, most preferred are linear saturated alkyl groups,
R₃ is selected from the group consisting of alkyl groups with 1 to 12, preferably 1 to 10, more preferred 1 to 7, even more preferred 1 to 6, especially preferred 1 to 5 and most preferred 1 to 4 carbon atoms, aryl groups with 6 to 14, preferably 6 to 12, and most preferred 6 to 10 carbon atoms, and aralkyl groups with 7 to 14, preferably 7 to 12, and most preferred 7 to 10 carbon atoms, wherein the alkyl groups may be linear, branched, cyclic, saturated or unsaturated, most preferred linear and saturated,
R⁴ is selected from the group consisting of alkyl groups with 3 to 12, preferably 3 to 10, more preferred 3 to 7, most preferred 4 to 6 carbon atoms, aryl groups with 6 to 14, preferably 6 to 12, and most preferred 6 to 10 carbon atoms, and aralkyl groups with 7 to 14, preferably 7 to 12, and most preferred 7 to 10 carbon atoms, wherein the alkyl groups may be linear, branched, cyclic, saturated or unsaturated, most preferred linear and saturated, and
X is selected from the group consisting of halide, preferably chloride and/or bromide, hydrogen sulfate, alkyl sulfate, preferably methylsulfate and ethylsulfate, carbonate, hydrogen carbonate, carboxylate, preferably acetate, or hydroxide.

In a first preferred embodiment the catalyst is a quaternary ammonium salt having the general structure R₁ R₂ R₃ R₄ NX wherein R₁ to R₄ are defined as described before and are selected such that the sum of carbon atoms of the ammonium cation is 6 to 14, preferably 7 to 14, more preferred 8 to 13.

In a second preferred embodiment the catalyst is a quaternary ammonium salt having the general structure R₁ R₂ R₃ R₄ NX wherein R₁ to R₄ are defined as described before and are selected such that the sum of carbon atoms of the ammonium cation is 15 to 30, preferably 15 to 28, more preferred 15 to 24, even more preferred 16 to 22 and most preferred 16 to 20.

In a third preferred embodiment the catalyst is a quaternary ammonium salt having the general structure R₁ R₂ R₃ R₄ NX wherein R₁ to R₄ and X are defined as described before and are selected such that the sum of carbon atoms of the ammonium salt is 6 to 14, preferably 7 to 14, more preferred 8 to 13.

In a fourth preferred embodiment the catalyst is a quaternary ammonium salt having the general structure R₁ R₂ R₃ R₄ NX wherein R₁ to R₄ and X are defined as described before and are selected such that the sum of carbon atoms of the ammonium salt is 15 to 30, preferably 15 to 28, more preferred 15 to 24, even more preferred 16 to 22 and most preferred 16 to 20.

Most preferred quaternary ammonium salts appropriate for use as the activating agent in the process of this invention include tetrabutylammounium hydrogensulfate, benzyltrimethylammonium chloride, tributyl methyl ammonium chloride and trioctyl methyl ammonium methyl sulphate.

The other class of activating agents useful in the practice of this invention includes organic sulfonates (i.e., organic compounds containing at least one sulfonate functional group). Such substances have the general formula

R-SO₃M
wherein R is a linear, branched, cyclic saturated or unsaturated alkyl group, an aryl group, or alkyl aryl group containing at least 7 carbon atoms and M is alkali metal (e.g., sodium, potassium), alkaline earth metal (e.g., calcium, barium, magnesium), or ammonium (NH₄, NHR₃, NH₂R₂, NH₃R), where M may also be hydrogen provided sufficient strong base is present during the hydrolysis reaction to convert the organic sulfonate into its salt (anionic) form and R is an organic moiety such as methyl or ethyl). Organic sulfonates are described in Cahn et al., “Surfactants and Detersive Systems”, in Encyclopedia of Chemical Technology, Vol. 22, pp. 347-360 (1983) and Mccutcheon, Synthetic Detergents, (1950) pp. 120-151. Preferably organic sulfonate selected from the group consisting of alkyl aryl sulfonates, alpha-olefin sulfonates, petroleum sulfonates and naphthalene sulfonates are used.

Since it is preferred to operate the process of the invention at temperatures as low as possible, it is preferred to use quaternary ammonium salt as the activating agent. Preferably the polyurethane is reacted with water and the organic amine base or with water, the organic amine base and the phase transfer catalyst in the process of the invention at a temperature of from 80° C. to 200° C., preferably 90° C. to 180° C., more preferred 95° C. to 170° C. and most preferred 100° C. to 160° C. If the temperature is too low, the yields are insufficient. Too high temperatures are inefficient from an economic point of view and might cause side reactions, forming unwanted by-products.

Preferably the polyurethane is reacted with water and the organic amine base or and with water and the organic amine base and the phase transfer catalyst for 1 minute to 30 hours, preferably 1 minute to 24 hours, more preferred 5 minutes to 20 hours, even more preferred 10 minutes to 20 hours, particular preferred 20 minutes to 18 hours, especially preferred 30 minutes hours to 18 hours and most preferred 30 minutes to 16 hours.

While water functions as a reactant in the desired polyurethane hydrolysis reaction and thus does not need to be present in stoichiometric excess relative to the urethane functional groups in the polymer to be hydrolyzed, it will generally be desirable to utilize a substantial quantity of water in order that it may conveniently serve as a reaction medium and solvent or carrier for the strong base and activating agent. For these reasons, the water is preferably present in condensed (liquid) form. Typically, the weight ratio of polyurethane to water is from 10:1 to 1:15, preferably from 5:1 to 1:15 and more preferred from 3:1 to 1:15.

To improve phase separation after completion of the hydrolysis it is preferred to use a solution of one or more inorganic salt(s) in water instead of pure water in the process of the invention. More preferred the inorganic salts are selected from the group consisting of ammonium or alkali metal (bi)-carbonates, (bi)-sulfates, phosphates, hydrogen phosphates, hydroxides, carboxylates and mixtures thereof, and even more preferred the aqueous solution of the one or more inorganic salts in water comprises in sum of from 10 to 60 wt. % inorganic salts, particular preferred in sum of from 20 to 50 wt. % inorganic salts and most preferred in sum of from 30 to 45 wt. % inorganic salts, in each case the wt. % relates to the weight of the aqueous solution of the inorganic base, or is a saturated solution.

The hydrolysis can be carried out at atmospheric pressure, although super-atmospheric pressures may be employed, if desired, preferably the reaction is carried out at atmospheric pressure or a pressure of 1 to 10 bar, more preferred 1 to 5 bar. Optionally, a water-miscible or water-immiscible solvent such as alcohol, ketone, ester, ether, amide, sulfoxide, halogenated hydrocarbon, aliphatic hydrocarbon, or aromatic hydrocarbon may be present in the reaction mixture to facilitate the hydrolysis process or to aid in recovering the reaction products.

The hydrolysis reaction may be carried out as a batch, continuous, or semi-continuous process in any appropriate vessel or other apparatus (for example, a stirred tank reactor or screw extruder). It will generally be preferred to agitate or stir the reaction components so as to assure intimate contact, rapid hydrolysis rates, and adequate temperature control.

The process of the present invention is beneficial compared to prior art processes because the hydrolysis reaction can be carried out as one step process. It is preferred that the polyurethane is not subjected to a reaction with a pure amine or with pure water before being subjected to hydrolysis with water and the organic amine base or with water, the organic amine base and the phase transfer catalyst, i.e. before being contacted simultaneously with water and the organic amine base or with water, the organic amine base and the phase transfer catalyst. “Pure” amine means PU being contacted with amine but not simultaneously with water. An example of such process is aminolysis as the processes disclosed in EP 0 990 674 A2 and DE 42 17 524 A1 wherein in a first step PU is reacted with pure amine but not simultaneously with water. “Pure” water means that the PU being contacted with water but not simultaneously with an amine.

The inventors surprisingly found out that it is not necessary to conduct a two-step process as the disclosed in EP 0 990 674 A2 and DE 42 17 524 A1 to efficiently recycle PU. According to the findings of the inventors a one step process is beneficial in view of room-time yield. No low molecular glycol or amino alcohol or alcohol is necessary and the inventive one step process can be operated continuously. It is thus, preferred that the reaction mixture during the inventive hydrolysis contains a glycol and/or an amino alcohol and/or an aliphatic alcohol in sum to a maximum content of 15 wt. %, preferably 0 to 10 wt. %, more preferred 0 to 5 wt. %, particular preferred 0 to 1 wt. %, in each case the wt. % are based on the total weight of the PU in the reaction mixture. Most preferred the reaction mixture does not comprise a glycol and/or an amino alcohol and/or an aliphatic alcohol during the inventive hydrolysis.

It is further preferred that the process of the invention does not comprise steps of isolating of reaction products of the organic amine base and the polyurethane followed by a subsequent hydrolysis of the isolated reaction products.

The organic amine base is separated by distillation or extraction or a membrane filtration which is different from filtration of a precipitate, more preferred organic solvent nanofiltration, most preferred by distillation or extraction from the active hydrogen containing polyether, preferably polyether polyol, and the organic polyamines formed during polyurethane hydrolysis. Preferably, after completion of the hydrolysis step, the organic amine base is separated from the reaction mixture by distillation or extraction or membrane filtration, more preferred organic solvent nanofiltration, more preferred it is separated by distillation or extraction, most preferred it is separated by distillation.

The separated organic amine base is preferably re-used in the process of the invention as catalyst. Separation of the organic amine base from the reaction products of the hydrolysis before the reaction products are separated from each other increases process efficiency and is an important step for a continuous process. To allow separation of the organic amine base from the reaction products of the hydrolysis via the preferred distillation method, it is particular preferred that the organic amine base having a boiling point below that of at least one, preferably more than one, more preferred all organic polyamines obtained as product of the polyurethane hydrolysis.

Precipitation methods as disclosed in CN110105621 are not suitable for continuous operation. The precipitation step causes an interruption of the process. The reaction mixture must be left to stand until precipitation is completed. Thus, the process disclosed in CN110105621 can only be operated batch-wise.

The active hydrogen containing polyether, organic polyamines, chain extenders, and curatives produced in the hydrolysis may be separated and recovered from the crude reaction mixture or preferred from the reaction mixture obtained after separation of the organic amine base, using any suitable method or combination of methods known in the art such as for example extraction (using water-immiscible organic solvents as the extractant, for example), distillation, precipitation, filtration.

The recovered active hydrogen containing polyether, in particular polyether polyols obtained in the process of the present invention are of excellent quality. The inventors found out that they can be used to produce polyurethane foams of high quality even without addition of virgin polyether polyol. This is a significant improvement compared to the polyurethane depolymerization processes of the prior art.

The recovered polyamines can be converted to organic polyisocyanates by conventional processes and similarly employed as components of polyurethanes.

Without further elaboration, it is believed that one skilled in the art can, using the preceding description, utilize the present invention of its fullest extent. The following examples, therefore, are to be considered as merely illustrative and not limitative of the claims or remainder of the disclosure in any.

### EXAMPLE 1-8

In Examples 1 to 8 polyurethane was hydrolyzed according to the invention with an organic amine base as follows:

A reactor from Parr instrumental company equipped with a PTFE liner and a mechanical stirrer, was charged with 25 g of compressed polyurethane foam pieces (ca. 1 cm×1 cm) and 75 g of the organic amine base was added. Thereafter the reactor was closed and heated to the operating temperature. After the desired reaction time was over the mixture was allowed to cool down, the reactor was opened, and the reaction mixture was transferred into a round-bottom flask. The reaction mixture was a green colored oily solution; this mixture was treated directly with cyclohexane.

Layers were separated and an acidic wash of the organic phase was performed twice with 0.4M aqueous HCl solution. The organic phase was dried over magnesium sulfate and the solvent was removed to give pure polyol as determined by NMR. The water layer was extracted with warm toluene to obtain the amine after drying and removal of solvent.

The used organic amine base, its amounts, reaction time and temperature as well as yields of the recovered polyether polyol and amine are given in Table 1.

TABLE 1

| | | | | | | Yield |
|---|---|---|---|---|---|---|
| | | | Base | Reaction | Reaction | Polyether- |
| | | | concen- | Temperature | Time | polyol |
| Example | Base* | Base category | tration | [° C.] | [hours] | [%] |
| 1 | 2-(2-dimethyl- | Base according | 2M aq. | 150 | 14 | 69.79 |
| | amino | to Formula (5) | Solution | | | |
| | ethyloxy) | with R¹ and R² = | | | | |
| | ethyl- | ethylene, all | | | | |
| | dimethyl- | R³ and R⁴ = | | | | |
| | amine | methyl, x = 1; | | | | |
| | | y = 1 and z = 2 | | | | |
| 2 | 2-(2-dimethyl- | Base according | 4M aq. | 150 | 14 | 73.05 |
| | amino | to Formula (5) | Solution | | | |
| | ethyloxy) | with R¹ and R² = | | | | |
| | ethyl- | ethylene, all | | | | |
| | dimethyl- | R³ and R⁴ = | | | | |
| | amine | methyl, x = 1; | | | | |
| | | y = 1 and z = 2 | | | | |
| 3 | Diisopropa- | Base according | 2M aq. | 150 | 14 | 94.00 |
| | nol-tetra- | to Formula (4) | Solution | | | |
| | methyl- | with all R² = | | | | |
| | propylene | propylene, four | | | | |
| | tetramine | R³ = methyl to | | | | |
| | | form two | | | | |
| | | dimethylamino | | | | |
| | | groups two R³ = | | | | |
| | | (iPrOH) to form | | | | |
| | | one diisopropa- | | | | |
| | | nolamino group | | | | |
| | | to Formula (6) | | | | |
| 4 | Mono- | Base according | 2M aq. | 150 | 14 | 78.83 |
| | DMAPA-Urea | to Formula (6) | Solution | | | |
| | mixed with | with R⁵ = | | | | |
| | dipropylene | propylene, all | | | | |
| | glycol | R⁶ = methyl, | | | | |
| | | all R⁷ = H, | | | | |
| | | a = 1, b = 1, | | | | |
| | | c = 1, d = 1 | | | | |
| 5 | Tetramethyl- | Base according | 2M aq. | 150 | 14 | 72.49 |
| | guanidine | to Formula (7) | Solution | | | |
| | | with all R⁷ = | | | | |
| | | methyl c = 2, | | | | |
| | | d = 0 | | | | |
| 6 | Tetramethyl- | Base according | 4M aq. | 150 | 14 | 88.51 |
| | guanidine | to Formula (7) | Solution | | | |
| | | with all R⁷ = | | | | |
| | | methyl c = 2, | | | | |
| | | d = 0 | | | | |
| 7 | 1,5-Diaza- | bicyclic, non- | 2M aq. | 150 | 14 | 60.00 |
| | bicyclo[4.3.0] | aromatic, | Solution | | | |
| | non-5-ene | nitrogen | | | | |
| | | comprising | | | | |
| | | organic base | | | | |
| 8 | 1,8,-Diaza- | bicyclic, non- | 2M aq. | 150 | 14 | 54.00 |
| | bicyclo- | aromatic, | Solution | | | |
| | undecene | nitrogen | | | | |
| | | comprising | | | | |
| | | organic base | | | | |

*Purity of the bases are ≥90%, more preferred ≥97% purity due to process inherent side products

Examples 1 to 8 show that polyurethanes can effectively be hydrolyzed using the organic amine bases of the invention at temperatures far below 200° C. Increasing the base concentration increases the polyol yield.

### Example 9-15

In examples 9 to 15 polyurethane foam was hydrolyzed according to the invention with an organic amine base and a quaternary ammonium salt as phase transfer catalyst as follows:

A reactor from Parr instrumental company equipped with a PTFE liner and a mechanical stirrer, was charged with 25 g of compressed polyurethane foam pieces (ca. 1 cm×1 cm) and 75 g of the organic amine base was added. Thereafter a phase transfer catalyst was added, the reactor was closed and heated to the operating temperature. After the desired reaction time was over the mixture was allowed to cool down, the reactor was opened and the reaction mixture was transferred into a round-bottom flask.

The reaction mixture was a green colored oily solution; this mixture was treated directly with cyclohexane.

Layers were separated and an acidic wash of the organic phase was performed twice with 1N aqueous HCl solution. The organic phase was dried over magnesium sulfate and the solvent was removed to give pure polyol as determined by NMR. The aqueous layer was extracted with warm toluene to obtain the amine after drying and removal of solvent.

The used base solution and catalyst, their amounts, reaction time and temperature as well as yields of the recovered polyether polyol and amine are given in Table 2

TABLE 2

| | | | | | | Yield |
|---|---|---|---|---|---|---|
| | | | | Reaction | Reaction | Polyether- |
| | | | | Temperature | Time | polyol |
| Ex. | Base* | Base category | Catalyst** | [C.] | [hours] | [%] |
| 9 | Tetra- | Base according to | TBAHS | 130 | 14 | 89.82 |
| | methyl- | Formula (7) with | (5 wt. % | | | |
| | guanidine | all R⁷ = methyl | in water) | | | |
| | | c = 2, d = 0 | | | | |
| 10 | 1,8- | bicyclic, non-aromatic, | TBAHS | 150 | 14 | 67.40 |
| | Diaza- | nitrogen comprising | (5 wt. % | | | |
| | bicyclo- | organic base | in water) | | | |
| | undecene | | | | | |
| 11 | N,N- | Base according to | TBAHS | 150 | 14 | 78.52 |
| | Dimethyl- | Formula (5) with R² = | (5 wt. % | | | |
| | amino- | ethylene, all R³ = | in water) | | | |
| | ethyl)- | methyl, one R⁴ = | | | | |
| | N-methyl- | methyl, one R⁴ = 2- | | | | |
| | amino- | hydroxyethyl x = 0; | | | | |
| | ethanol | y = 1 and z = 2 | | | | |
| 12 | Mono- | Base according to | TBAHS | 150 | 14 | 96.73 |
| | DMAPA- | Formula (6) with R⁵ = | (5 wt. % | | | |
| | Urea | propylene, all R⁶ = | in water) | | | |
| | mixed with | methyl, all R⁷ = H, | | | | |
| | dipropylene | a = 1, b = 1, | | | | |
| | glycol | c = 1, d = 1 | | | | |
| 13 | N,N- | Base according to | TBAHS | 150 | 14 | 60.00 |
| | Dimethyl- | Formula (5) with R¹ | (5 wt. % | | | |
| | amino- | and R² = ethylene, all | in water) | | | |
| | ethyl- | R³ = methyl, one R⁴ = | | | | |
| | oxyethyl- | methyl, one R⁴ = 2- | | | | |
| | N-methyl- | hydroxyethyl x = 1; | | | | |
| | amino- | y = 1 and z = 2 | | | | |
| | propanol | | | | | |
| 14 | Bis- | Base according to | TBAHS | 150 | 14 | 69.10 |
| | (Dimethyl- | Formula (4) with all | (5 wt. % | | | |
| | amino)-N- | R² = propylene, four | in water) | | | |
| | propyl- | R³ = methyl to form | | | | |
| | propylene | two dimethylamino | | | | |
| | diamine | groups two R³ = H to | | | | |
| | | form one amino group | | | | |
| 15 | Diisopro- | Base according to | TBAHS | 150 | 14 | 87.31 |
| | panol- | Formula (4) with all | (5 wt. % | | | |
| | tetra- | R² = propylene, four | in water) | | | |
| | methyl- | R³ = methyl to form | | | | |
| | propylene | two dimethylamino | | | | |
| | tetramine | groups two R³ = (iPrOH) | | | | |
| | | to form one diiso- | | | | |
| | | propanolamino group | | | | |

*Purity of the bases are ≥90%, more preferred ≥97% purity due to process inherent side products
**TBAHS = Tetrabutylammouniumhydrogensulfate (C = 16)

Comparison of Example 12 to Example 4 and of Example 9 to Example 5 show that the polyether polyol yield can be further increased if an organic amine base of the invention is used together with a catalyst of the invention for hydrolysis of the polyurethane. Excellent yields can be achieved even at moderate reaction temperatures and under non-corrosive conditions.

### EXAMPLE 16

Example 1 was repeated with the following modifications:

A crude reaction mixture, obtained from the hydrolyzation of 500 g of PU foam with 700 g of 2-(2-dimethylamino ethyloxy) ethyl-dimethyl-amine base and 200 g of water at 160° C. was, after cooling to room temperature, directly subjected to a distillation to remove residual water and the aliphatic amine base, i.e. 2-(2-dimethylamino ethyloxy) ethyl-dimethyl-amine. As first distillation fraction water, as second fraction a small mixed fraction of amine/water and as third fraction pure 2-(2-dimethylamino ethyloxy) ethyl-dimethyl-amine was obtained. 2-(2-dimethylamino ethyloxy) ethyl-dimethyl-amine was recovered in 98% yield and re-used in further polyurethane hydrolysis reactions.

The residue of the distillation consisted of a mixture of aromatic amine and polyol, i.e. the two PU hydrolysis products; only traces of 2-(2-dimethylamino ethyloxy) ethyl-dimethyl-amine were detected. The residue was treated with cyclohexane to extract polyol from the aromatic amine. The hexane layer was washed with 1N HCl and dried over magnesium sulfate to yield 314 g of polyol and 90 g of TDA amine.

Example 13 shows that the reaction products of the inventive hydrolysis as well as the amine base catalyst can be separated from each other and recovered efficiently, in good yield and high purity via a combination of distillation and extraction. This is a significant improvement compared to long lasting and very water consuming precipitation processes as used in the prior art, for example in CN 110105621 B1.

### Comparative Example 1

Applicants reproduced Example 3 of CN110105621 B and found several unknown signals of side products which were not found in the process of the invention. In detail:

1.6 g of the same flexible PU foam pieces (ca. 1 cm×1 cm) as used in the inventive Examples outlined above were decomposed using 3.2 g of 1,4-dimethylpiperazine in 80.0 g of an 20% aq. MeOH solution at 250° C. for 1 hour. Then the mixture was allowed to cool to room temperature and afterwards 42.4 g of water were added. After one hour the mixture was transferred to a separatory funnel. A two-phase system with a transparent phase and a thick oily black material accumulating at the base was obtained. The mixture was filtered to get 410 mg of a dark deposit on the filter paper. The residue was dissolved in DCM and then concentrated to get a dark brown pasty mass. The filtrate consisted of a mixture of aromatic amine, polyol and 1,4-dimethylpiperazine. The filtrate was extracted with n-hexane to give polyol together with residual TDA amine and an aqueous phase.

A sample of the aqueous phase was concentrated and analyzed: here, a mixture of aromatic amine, 1,4-dimethylpiperazine and traces of unknown signals were found.

The rework of Example 3 of CN110105621 B shows that difficult to handle phases were obtained. In addition side products were formed that were not visible with the process of the invention. Without being bond to the theory, applicants believe that the fact that the organic amine base is not separated from the other components of the reaction mixture before precipitation takes place and the reaction mixture is left to stand in CN110105621 B causes the formation of the side products. Additional purification measures would be necessary to recover pure products, if at all possible.

### Comparative Example 2

Applicants reproduced Example 4 of CN110105621 B and again found several unknown signals of side products which were not found in the process of the invention. In detail:

1.2 g of flexible PU foam pieces (ca. 1 cm×1 cm), same as in the inventive Examples outlined above and as in Comparative Example 1, were decomposed using 1.5 g of 1,4-dimethylpiperazine and 132 g of the aqueous phase from Comparative Example 1 at 180° C. for one hour. Then 300 g of water were added mixed well and the mixture was left for stand for an additional hour. Dark brown tar like material had settled at the bottom of brown hazy solution. The solution was filtered to separate the brown deposit which was then collected by dissolving in DCM and concentrated to obtain 250 mg of a brown paste. 1H NMR showed majorly polyol signals and traces of amine.

A sample from the filtrate was concentrated and analyzed to show a mixture of amine, polyol and 1,4-dimethylpiperazine. The filtrate was extracted with ethyl acetate (3×100 mL) and concentrated to obtain 880 mg of a brown oil. 1H NMR of the brown oil showed a mixture of polyol and amine signals.

A sample from the aqueous phase was concentrated to give a mixture of TDA, 1,4-dimethylpiperazine and traces of unknown signals.

Comparative Example 2 shows that if the aqueous phase obtained in Comparative Example 1 is re-used as hydrolyzing reagent in the process of D3 even more complex product mixtures were obtained. No product or other component of the reaction mixture could be isolated in pure form. The unwanted side products obtained in Comparative Example 1 accumulate in the process if the water phase is recycled. Thus, the process disclosed in D3 is not suitable for continued operation with re-use of the catalyst.

### Application Tests

### Production of Hot-Cure Flexible PU Foams (Flexible Slabstock Foam)

For the performance testing of the recycled polyols, the hot-cure flexible PU foam formulation specified in Table 3 can be used.

TABLE 3
Formulation for hot-cure flexible PU foam production.

| Formulation 1 | Parts by mass (pphp) | |
|---|---|---|
| Polyol¹⁾ | 100 | parts |
| Water | 4.00 | parts |
| KOSMOS ® T9²⁾ | 0.20 | parts |
| DABCO ® DMEA³⁾ | 0.15 | parts |
| TEGOSTAB ® BF2370⁴⁾ | 1.0 | part |

| | |
|---|---|
| Desmodur ® T 80⁵⁾ | Variable, Constant Index of 105 |

¹⁾Polyol 1: Standard virgin polyol Arcol ® 1104 available from Covestro, this is a glycerol-based polyether polyol having an OH number of 56 mg KOH/g and an average molar mass of 3000 g/mol or inventive recycled polyols or non-inventive recycled polyol. The recycled polyols are obtained by chemical recycling from flexible polyurethane foams. The recycled polyols were obtained by the procedures described in the following paragraphs.
²⁾KOSMOS ® T9, available from Evonik Industries: tin(II) salt of 2-ethylhexanoic acid.
³⁾DABCO ® DMEA: dimethylethanolamine, available from Evonik Industries. Amine catalyst for production of polyurethane foams.
⁴⁾Polyether-modified polysiloxane, available from Evonik Industries.
⁵⁾Tolylene diisocyanate T 80 (80% 2,4 isomer, 20% 2,6 isomer) from Covestro, 3 mPa · s, 48% NCO, functionality 2.

### Production of Recycled Polyols

### Recycled Polyol 1 (Non-Inventive)

The non-inventive recycled polyol 1 was produced following a procedure published by H&S Anlagentechnik in 2012: https://www.dbu.de/OPAC/ab/DBU-Abschlussbericht-AZ-29395.pdf

A Reactor from Parr instrumental company equipped with a glass in liner and a mechanical stirrer, was charged with 300.2 g of compressed polyurethane foam pieces (ca. 1 cm×1 cm). The used polyurethane foam was produced according to Formulation 1, Table 3 by using the conventional polyol Arcol®1104.

152.64 g of the polyol Arcol®1104, 75.63 g phthalic acid and 11.97 g hydrogen peroxide (30 wt % in water) were added to the foam pieces. The reaction mixture was heated to 250° C. inner-temperature.

The reaction was kept under this condition for 5 hours at an inner-temperature between 237° C. and 256° C. After the heating was stopped the second portion of 140.63 g Arcol®1104 was added at 160° C. under nitrogen atmosphere. At 80° C. the reaction mixture was decanted and then cooled down to room temperature. The cooled and decanted reaction mixture was used as non-inventive recycled polyol 1. The process can be repeated to generate a sufficient quantity recycled polyol for foaming experiments.

### Recycled Polyol 2 (Inventive)

The inventive recycled polyol 2 from inventive Example 5 was used.

### Recycled Polyol 3 (Inventive)

The inventive recycled polyol 3 from inventive Example 10 was used.

### General Procedure for Production of the Foam Samples

For each foaming test 300 g of polyol are used; the other formulation constituents are recalculated accordingly. 1.00 part of a component denoted 1.00 g of this substance per 100 g of polyol for example.

The foaming is carried out in the so-called manual mixing process. Formulation 1 as specified in Table 3 are used. To this end, a paper cup is charged with the different polyols, the respective amine catalyst, the tin catalyst tin (II) 2-ethylhexanoate, water and a foam stabilizer, and the contents are mixed at 1000 rpm for 60 seconds with a disc stirrer. After the first stirring the isocyanate (TDI) is added to the reaction mixture and stirred at 2500 rpm for 7 s and then the reaction mixture is immediately transferred into a paper-lined box (30 cm×30 cm base area and 30 cm height). After being poured in, the foam rises in the foaming box. In the ideal case, the foam blow off on attainment of the maximum rise height and then fall back slightly. This opens the cell membranes of the foam bubbles and an open-pore cell structure of the foam can be obtained. Defined foam bodies can be cut out of the resulting hot-cure flexible PU foam blocks and can be analyzed further.

### Characterization of the Flexible PU Foams:

The flexible polyurethane foams produced can be assessed according to the following foam properties a) to j):
a) Fallback of the foam after the end of the rise phase (=settling): The settling, or the further rise, is found from the difference of the foam height after direct blow-off and after 3 minutes after foam blow-off. The foam height is measured at the maximum in the middle of the foam crest by means of a needle secured to a centimeter scale. A positive value here describes the settling of the foam after blow-off; a negative value correspondingly describes further rise of the foam after the blow off.
b) Foam height: The height of the freely risen foam formed after 3 minutes. Foam height is reported in centimeters (cm).
c) Rise time: The period of time between the end of mixing of the reaction components and the blow-off of the polyurethane foam. The rise time is reported in seconds(s).
d) Porosity by dynamic pressure measurement: The gas permeability of the foam was determined in accordance with DIN EN ISO 4638:1993-07 by a dynamic pressure measurement on the foam. The dynamic pressure measured was reported in mm water column, and lower dynamic pressure values characterize a more open foam. The values were measured in the range from 0-300 mm water column. The dynamic pressure was measured by means of an apparatus comprising a nitrogen source, reducing valve with pressure gauge, flow regulating screw, wash bottle, flow meter, T-piece, applicator nozzle and a graduated glass tube filled with water. The applicator nozzle has an edge length of 100×100 mm, a weight of 800 g, an internal diameter of the outlet opening of 5 mm, an internal diameter of the lower applicator ring of 20 mm and an external diameter of the lower applicator ring of 30 mm. The measurement is carried out by setting the nitrogen admission pressure to 1 bar by means of the reducing valve and setting the flow rate to 480 l/h. The amount of water in the graduated glass tube is set so that no pressure difference is built up and none can be read off. For measurement on the test specimen having dimensions of 250×250×50 mm, the applicator nozzle is laid onto the corners of the test specimen, flush with the edges, and also once onto the (estimated) middle of the test specimen (in each case on the side having the greatest surface area). The result is read off when a constant dynamic pressure has been established. The final result is calculated by forming the average of the five measurements obtained.
e) Number of cells per cm (cell number): This is determined visually on a cut surface (measured to DIN EN 15702:2009-04).
f) Compression hardness CLD, 40% to DIN EN ISO 33861:1997+A1:2010. The measured values are reported in kilopascals (kPa).
g) Constant Deflection Compression Set (also commonly called compression set)

Five test specimens each of size 5 cm×5 cm×2.5 cm were cut out of the finished foams. The starting thickness was measured. Compression set was measured no earlier than 72 h after production in accordance with DIN EN ISO 1856:2018-11. The test specimens were placed between the plates of the deforming device and were compressed by 90% of their thickness (i.e. to 2.5 mm). Within 15 minutes, the test specimens were placed into an oven at 70° C. and left therein for 22 h. After this time, the apparatus was removed from the oven, the test specimens were removed from the apparatus within 1 min, and they were placed on a wood surface. After relaxation for 30 min, the thickness was measured again and the compression set was calculated and results are reported as a percentage of the original thickness:

DVR=(d0−dr)/d0×100%
h) Tensile strength and elongation at break to DIN EN ISO 1798:2008-04. The measurements of tensile strength are reported in kilopascals (kPa), and those of elongation at break in percent (%).
i) Rebound resilience to DIN EN ISO 8307:2008-03. The measurements are reported in percent (%).
j) Odor testing of the resulting foams. The finished foams were packed in odor-neutral plastic bags and stored under airtight conditions. For the odor assessment of the foam, cubes measuring 10 cm×10 cm×10 cm were cut out and transferred to jars with a volume of 1 liter, from which the samples were smelled. The jars were closed with a screw lid. The odor test took place after storing the jars for 24 hours at 22° C. The odor test was assessed by a panel of 13 trained odor testers. They were questioned here about the intensity of the odor, a low odor level was rated+, moderate odor++, and high odor+++.

### Results of the Foaming Experiments

The results of the influence of the recycled polyols according to the invention on foaming process and foam physical properties of the resulting hot-cure flexible PU foams are compiled in the tables below. Hot-cure flexible PU foams were produced following formulation 1, Table 3 with a standard virgin polyol, recycled polyol not inventive and with the inventive recycled polyols 2 and 3.

TABLE 4
Foaming results and foam physical properties of the foams with use
of different types of polyols according to formulation 1, Table 3.

| Foam Sample | #1 | #2 | #3 | #4 |
|---|---|---|---|---|
| Arcol ®1104, OHN 56, | 100 | | | |
| Reference | | | | |
| Recycled Polyol 1 (non- | | 100 | | |
| inventive), OHN 82 | | | | |
| Recycled Polyol 2 | | | 100 | |
| (inventive), OHN 57 | | | | |
| Recycled Polyol 3 | | | | 100 |
| (inventive), | | | | |
| OHN 56 | | | | |
| Index | 105 | 105 | 105 | 105 |
| Rise time (s) | 120 | — | 118 | 139 |
| Rise height (cm) | 24.2 | — | 24.4 | 23.4 |
| Settling (cm) | 0.0 | — | 0.0 | 0.1 |
| Cells (per cm) | 13 | — | 13 | 13 |
| Porosity | 14 | — | 17 | 12 |
| (mm water column) | | | | |
| Hardness CLD 40% | 3.8 | — | 3.8 | 3.7 |
| compression (kPa) | | | | |
| Elongation (%) | 154 | — | 135 | 155 |
| Tensile Strength (kPa) | 120 | — | 102 | 121 |
| Ball Rebound (%) | 44.7 | — | 43.3 | 42.5 |
| Compression Set | 7 | — | 7.2 | 8.8 |
| 90% 22 h at 70° C. (%) | | | | |
| Odour | + | | +++ | ++ |
| Remarks | Standard | Collapse | Standard | Standard |
| | foam | | foam | foam |

In general, the foaming results in Table 4 show that replacing the standard virgin polyol Arcol®1104 by the inventive recycled polyol 2 and 3 allows the production of flexible PU foam with comparable foaming processing characteristics to the reference foam #1.

For recycled polyol 2, the foam physical properties including porosity, cell count, ball rebound and compression set of the inventive foam #3 are comparable to the reference foam #1. Only small differences are observed for the physical properties with respect to elongation and tensile strength when using the inventive recycled polyol 2 compared to the reference foam #1.

For recycled polyol 3, the foam physical properties including porosity, cell count, ball rebound, elongation and tensile strength of the inventive foam #4 are comparable to the reference foam #1. Only small differences are observed for the physical properties with respect to compression set when using the inventive recycled polyol 3 compared to the reference foam #1. Additionally, during foam production, the rise time is slightly longer in comparison to the rise time for the reference polyol and the recycled polyol 2.

On the contrary it was not possible to produce any reasonable foam by using 100 pphp of the non-inventive recycled polyol 1, the use of this recycled polyol in foam #2 caused the foam to collapse.

1. A method of hydrolyzing a polyurethane, the method comprising:
contacting said polyurethane with water in the presence of an organic amine base to yield an active hydrogen containing polyether, and an organic polyamine,
wherein
the organic amine base is an aliphatic amine, and
a reaction mixture comprising the polyurethane, water and the organic amine base is a stirred homogeneous or heterogeneous mixture, during hydrolysis, and
the organic amine base is separated from the active hydrogen containing polyether, and organic polyamine formed during polyurethane hydrolysis via distillation or extraction or membrane filtration.

2. The method according to claim 1,
comprising:
contacting the polyurethane with water in the presence of an organic amine base and a phase transfer catalyst.

3. The method of claim 1, wherein the organic amine base comprises one or more tertiary nitrogen atom(s) and/or has a boiling point below that of at least one organic polyamine obtained as product of the polyurethane hydrolysis.

4. The method of claim 1, wherein the organic amine base is at least one selected from the group consisting of
a base according to Formula (1)
(((R³)₂N—R²)—(O—R¹)ₓ)_{y}—N(R⁴)_{z} (1)
wherein
the R¹ groups in the molecule may be identical or different, the R² groups in the molecule may be identical or different, the R³ groups in the molecule may be identical or different, and the R⁴ groups in the molecule may be identical or different, and wherein
R¹ are identical or different and are independently from each other selected from the group consisting of linear or branched alkylene radicals with 1 to 10 carbon atoms,
R² are identical or different and are independently from each other selected from the group consisting of linear or branched alkylene or hydroxy alkylene radicals with 1 to 20 carbon atoms and if R² is hydroxy alkylene R² comprises 1 to 5 hydroxy group(s),
R³ are identical or different and are independently from each other selected from the group consisting of hydrogen, linear or branched alkyl groups with 1 to 6 carbon atoms, linear or branched hydroxy alkyl groups with 1 to 6 carbon atoms, (CH₂CH₂O)ᵤH, (CH₂CH₂CH₂O)ᵥH and (CH₂CH(CH₃)CH₂O)_{w}H, most preferred hydrogen, methyl, ethyl, propyl, isopropyl, hydroxymethyl, 2 hydroxyethyl, 3 hydroxypropyl, 2 hydroxypropyl, 2-hydroxyisopropyl,
R⁴ are identical or different and are independently from each other selected from the group consisting of hydrogen, linear or branched, cyclic or alicyclic alkyl groups with 1 to 20 carbon atoms, linear or branched hydroxy alkyl groups with 1 to 6 carbon atoms and cycloalkyl residues having 6 to 18 carbon atoms, (CH₂CH₂O)ᵤH, (CH₂CH₂CH₂O)ᵥH and (CH₂CH(CH₃)CH₂O)_{w}H, and wherein
u=1 to 14,
v=1 to 14,
w=1 to 14,
x=0 or 1,
y=0 to 3,
z=0 to 3, with the proviso that if z=3, at least one of R⁴ is not hydrogen,
y+z=3,
a base according to formula (2)
(((R⁶)₂N—R⁵)ₐ(H)_{b}N)_{d}—CZ—(N(R⁷)₂)_{c} (2)
wherein
the R⁵ groups in the molecule may be identical or different, the residues R⁶ groups in the molecule may be identical or different and the residues R⁷ groups in the molecule may be identical or different, and wherein
R⁵ are identical or different and are independently from each other selected from the group consisting of linear or branched alkylene radicals with 1 to 10 carbon atoms, wherein one or more CH₂ groups may be replaced by O to form ether bonds,
R⁶ are identical or different and are independently from each other selected from the group consisting of hydrogen, linear or branched alkyl groups with 1 to 6 carbon atoms, linear or branched hydroxy alkyl groups with 1 to 6 carbon atoms, (CH₂CH₂O)ᵤH, (CH₂CH₂CH₂O)ᵥH and (CH₂CH(CH₃)CH₂O)_{w}H,
R⁷ are identical or different and are independently from each other selected from the group consisting of hydrogen, linear or branched alkyl groups with 1 to 6 carbon atoms, linear or branched hydroxy alkyl groups with 1 to 6 carbon atoms, (CH₂CH₂O)ᵤH, (CH₂CH₂CH₂O)ᵥH and (CH₂CH(CH₃)CH₂O)_{w}H, and wherein
Z═O or NH,
u=1 to 14, preferably 1 to 6
v=1 to 14, preferably 1 to 6
w=1 to 14, preferably 1 to 6
a=0, 1 or 2,
b=0, 1 or 2,
a+b=2,
c=0, 1 or 2,
d=0, 1 or 2,
c+d=2,
cyclic or bicyclic, non-aromatic, nitrogen comprising organic base comprising 4 to 20 carbon atoms, and 1 to 4 nitrogen atoms, optionally the cyclic or bicyclic, non-aromatic, nitrogen comprising organic base comprises one or more O atoms and/or carries one or more substituents selected from the group consisting of linear or branched alkyl or alkenyl groups with 1 to 6 carbon atoms, linear or branched hydroxy alkyl groups with 1 to 6, carbon atoms, (CH₂CH₂O)ᵤH, (CH₂CH₂CH₂O)ᵥH and (CH₂CH(CH₃)CH₂O)_{w}H,
and mixtures thereof.

5. The method according to claim 4, wherein the organic amine base is at least one selected from the group consisting of
a trialkylamine according to Formula (3)
NR⁴R⁴′R⁴″ (3)
with R⁴, R⁴′, R⁴″ are identical or different and are independently selected from the group consisting of hydrogen, linear or branched, cyclic or alicyclic alkyl groups with 1 to 20 carbon atoms, linear or branched hydroxy alkyl groups with 1 to 6 carbon atoms and cycloalkyl residues having 6 to 18 carbon atoms, (CH₂CH₂O)ᵤH, (CH₂CH₂CH₂O)ᵥH and (CH₂CH(CH₃)CH₂O)_{w}H,
u=1 to 14,
v=1 to 14,
w=1 to 14,
with the provision at least one of R⁴, R⁴′ and
R⁴″ is not hydrogen,
a polyamine according to Formula (4)
((R³)₂N—R²)₃N (4),
wherein
R² are identical or different and are independently from each other selected from the group consisting of linear or branched alkylene or hydroxy alkylene radicals with 1 to 20 carbon atoms and if R² is hydroxy alkyl R² comprises 1 to 5 hydroxy group(s),
R³ are identical or different and are independently from each other selected from the group consisting of hydrogen, linear or branched alkyl groups with 1 to 6 carbon atoms, linear or branched hydroxy alkyl groups with 1 to 6 carbon atoms, (CH₂CH₂O)ᵤH, (CH₂CH₂CH₂O)ᵥH and (CH₂CH(CH₃)CH₂O)_{w}H,
u=1 to 14,
v=1 to 14,
w=1 to 14,
a polyamine according to Formula (5)
(((R³)₂N—R²)—(O—R¹)ₓ)_{y}—N(R⁴)_{z} (5)
wherein x=0 or 1, y=1 or 2 and z=1 or 2 and y+z=3 and wherein
R¹ are identical or different and are independently from each other selected from the group consisting of linear or branched alkylene radicals with 1 to 10 carbon atoms,
R² are identical or different and are independently from each other selected from the group consisting of linear or branched alkylene or hydroxy alkylene radicals with 1 to 20 carbon atoms and if R² is hydroxy alkyl R² comprises 1 to 5 hydroxy group(s),
R³ are identical or different and are independently from each other selected from the group consisting of hydrogen, linear or branched alkyl groups with 1 to 6 carbon atoms, linear or branched hydroxy alkyl groups with 1 to 6 carbon atoms, (CH₂CH₂O)ᵤH, (CH₂CH₂CH₂O)ᵥH and (CH₂CH(CH₃)CH₂O)_{w}H,
R⁴ are identical or different and are independently from each other selected from the group consisting of hydrogen, linear or branched, cyclic or alicyclic alkyl groups with 1 to 6 carbon atoms, linear or branched hydroxy alkyl groups with 1 to 6 carbon atoms, CH₂CH₂O)ᵤH, (CH₂CH₂CH₂O)ᵥH and (CH₂CH(CH₃)CH₂O)_{w}H,
u=1 to 14,
v=1 to 14,
w=1 to 14,
a polyaminoalkylurea according to Formula (6)
(((R⁶)₂N—R⁵)ₐ(H)_{b}N)_{d}—CO—(N(R⁷)₂)_{c} (6)
wherein
R⁵ are identical or different and are independently from each other selected from the group consisting of linear or branched alkylene radicals with 1 to 10 carbon atoms, wherein one or more CH₂ groups may be replaced by O to form ether bonds,
R⁶ are identical or different and are independently from each other selected from the group consisting of hydrogen, linear or branched alkyl groups with 1 to 6 carbon atoms, linear or branched hydroxy alkyl groups with 1 to 6 carbon atoms, (CH₂CH₂O)ᵤH, (CH₂CH₂CH₂O)ᵥH and (CH₂CH(CH₃)CH₂O)_{w}H,
R⁷ are identical or different and are independently from each other selected from the group consisting of hydrogen, linear or branched alkyl groups with 1 to 6 carbon atoms, linear or branched hydroxy alkyl groups with 1 to 6 carbon atoms, (CH₂CH₂O)ᵤH, (CH₂CH₂CH₂O)ᵥH and (CH₂CH(CH₃)CH₂O)_{w}H, and wherein
u=1 to 14,
v=1 to 14,
w=1 to 14,
a=0, 1 or 2,
b=0, 1 or 2,
a +b=2,
c=0, 1 or 2,
d=0, 1 or 2,
c+d=2,
an organic base comprising a guanidino group according to Formula (7)
(((R⁶)₂N—R₅)ₐ(H)_{b}N)_{d}—C(NH)—(N(R₇)₂)_{c} (7)
wherein
R⁵ are identical or different and are independently from each other selected from the group consisting of linear or branched alkylene radicals with 1 to 10 carbon atoms, wherein one or more CH₂ groups may be replaced by O to form ether bonds,
R⁶ are identical or different and are independently from each other selected from the group consisting of hydrogen, linear or branched alkyl groups with 1 to 6 carbon atoms, linear or branched hydroxy alkyl groups with 1 to 6; carbon atoms, (CH₂CH₂O)ᵤH, (CH₂CH₂CH₂O)ᵥH and (CH₂CH(CH₃)CH₂O)_{w}H,
R⁷ are identical or different and are independently from each other selected from the group consisting of hydrogen, linear or branched alkyl groups with 1 to 6 carbon atoms, linear or branched hydroxy alkyl groups with 1 to 6 carbon atoms, (CH₂CH₂O)ᵤH, (CH₂CH₂CH₂O)ᵥH and (CH₂CH(CH₃)CH₂O)_{w}H, and wherein
u=1 to 14,
v=1 to 14,
w=1 to 14,
a=0, 1 or 2,
b=0, 1 or 2,
a +b=2,
c=0, 1 or 2,
d=0, 1 or 2,
c+d=2,
and mixtures thereof.

6. The method of claim 2, wherein the phase transfer catalyst is a quaternary ammonium salt containing 6 to 30 carbon atoms and having the general structure R₁ R₂ R₃ R₄ NX wherein R₁, R₂, R₃, and R₄ are the same or different and are hydrocarbyl groups selected from the group consisting of alkyl, aryl, and arylalkyl and X is selected from the group consisting of halide, hydrogen sulfate, alkyl sulfate, carbonate, hydrogen carbonate, carboxylate, and hydroxide.

7. The method of claim 6, wherein R₁ and R₂ are the same or different and are alkyl groups with 1 to 12 carbon atoms, wherein the alkyl groups may be linear, branched, cyclic, saturated or unsaturated,
R₃ is selected from the group consisting of alkyl groups with 1 to 12 carbon atoms, aryl groups with 6 to 14 carbon atoms, and aralkyl groups with 7 to 14 carbon atoms, wherein the alkyl groups may be linear, branched, cyclic, saturated or unsaturated, and,
R₄ is selected from the group consisting of alkyl groups with 3 to 12 carbon atoms, aryl groups with 6 to 14 carbon atoms, and aralkyl groups with 7 to 14 carbon atoms, wherein the alkyl groups may be linear, branched, cyclic, saturated or unsaturated, and
X is selected from the group consisting of halide, hydrogen sulfate, alkyl sulfate, carbonate, hydrogen carbonate, acetate and hydroxide.

8. The method of claim 6, wherein R₁ to R₄ are selected such that the sum of carbon atoms in the quaternary ammonium cation is 6 to 14, or
R₁ to R₄ are selected such that the sum of carbon atoms in the quaternary ammonium cation is 15 to 30.

9. The method of claim 6, wherein R₁ to R₄ and X are selected such that the sum of carbon atoms in the quaternary ammonium salt is 6 to 14, or
R₁ to R₄ and X are selected such that the sum of carbon atoms in the quaternary ammonium salt is 15 to 30.

10. The method of claim 2, wherein the phase transfer catalyst is at least one organic sulfonate containing at least 7 carbon atoms selected from the group consisting of alkyl aryl sulfonates, alpha-olefin sulfonates, petroleum sulfonates and naphthalene sulfonates.

11. The method of claim 1, wherein after completion of the hydrolysis step, the organic amine base is separated from the reaction mixture by distillation or extraction.

12. The method of claim 1, further comprising:
separating and recovering the organic polyamine and/or active hydrogen containing polyether.

13. The method according to claim 12, wherein the organic polyamine and/or the active hydrogen containing polyether is separated from the reaction mixture obtained after the organic amine base has been separated.

14. The method of claim 1, wherein the polyurethane is foamed.

15. The method of claim 1, wherein the polyurethane is contacted with water and the organic amine base or with water, the organic amine and, optionally, the phase transfer catalyst,
at a temperature of from 80° C. to 200° C., and/or
for 1 minute to 30 hours, and/or
at atmospheric pressure or under pressure of 1 to 10 bar.

16. The method of claim 2, wherein at least 0.5 weight percent catalyst, based on the weight of the polyurethane is used.

17. The method of claim 1, wherein the weight ratio of the sum of the organic amine base to polyurethane is in the range of from 1:100 to 50:1.

18. The method of claim 1, wherein the polyurethane is not subjected to a reaction with a pure amine or pure water before being subjected to hydrolysis with water and the organic amine base or with water, the organic amine base and the catalyst.

19. The method of claim 1, wherein it does not comprise isolating of reaction products of the organic amine base and the polyurethane and subsequent hydrolysis of the isolated reaction products.

20. The method of claim 15, wherein the polyurethane is contacted with water and the organic amine base or with water, the organic amine and, optionally, the phase transfer catalyst,
at a temperature of from 100° C. to 160° C., and/or
for 30 minutes to 16 hours, and/or
at atmospheric pressure or under pressure of 1 to 5 bar.
